(19) United States
(12) Patent Application Publication
(10) Pub. No.: US 2023/0301605 A1
(43) Pub. Date: Sep. 28, 2023

(54) DIAGNOSTIC APPARATUS FOR NUCLEAR MEDICINE AND ESTIMATION METHOD OF ATTENUATION COEFFICIENT IMAGE

(71) Applicant: SHIMADZU CORPORATION, KYOTO-SHI (JP)

(72) Inventors: Suzuka Minagawa, Kyoto-shi (JP); Yoshiyuki Yamakawa, Kyoto-shi (JP)

(73) Assignee: SHIMADZU CORPORATION, KYOTO-SHI (JP)

(21) Appl. No.: 18/109,139

(22) Filed: Feb. 13, 2023

(30) Foreign Application Priority Data
Mar. 24, 2022 (JP) 2022-048284

(51) Int. Cl. A61B 6/03; G06T 11/00; G06T 7/60; G06V 10/74

(52) U.S. Cl. CPC A61B 6/037; G06T 11/008; G06T 7/60; G06V 10/761; G06T 2207/10104

(57) ABSTRACT

A diagnostic apparatus for nuclear medicine executes an attenuation correction processing with a high degree of precision relative to the radiation image and an estimation method of an attenuation coefficient image without irradiating radiation to an examination subject using an external radiation source. The diagnostic apparatus has a memory storage, a reconstruction element, a position deviation calculating element, a position correction element generating the corrected attenuation coefficient image, and a attenuation correction element using a corrected attenuation coefficient image HF and a concurrent counting data DK.

### CROSS REFERENCE TO RELATED APPLICATION

This application relates to, and claims priority from JP Ser. No.: JP2022-048284 filed Mar. 24, 2022, the entire contents of which are incorporated herein by reference.

### FIGURE FOR PUBLICATION

FIG. 6.

### BACKGROUND OF THE INVENTION

### Field of the Invention

The present invention relates to a diagnostic apparatus for nuclear medicine and an estimation method of attenuation coefficient image.

### Description of the Related Art

One example of the diagnostic apparatuses for nuclear medicine used in a medical site is a PET apparatus (Positron Emission Tomography). The PET apparatus detects a pair of annihilation radiations (γ-rays) that are emitted from the position of the radioactive pharmaceuticals. Specifically, a plurality of detectors that detect γ-rays is in place in the surrounding area of the examination subject, and when a pair of γ-rays is detected in an allotted time period (concurrently counted), such pairs of γ-rays are measured as effective signals. The measurement data of γ-rays obtained by such a measurement are called emission data, a radiation image (PET image) showing distribution of the radioactive pharmaceutical is obtained by conducting a reconstruction processing of the emission data.

As to the PET apparatus, a variety of data correction processings are needed to measure quantitatively the concentration of the radioactive pharmaceutical inside the examination subject. One example of typical data correction processing is an attenuation correction. A heterogeneous property of γ-ray attenuation corresponding to a position inside the examination subject is corrected by conducting the attenuation correction, so that the quantitative property of the radioactive concentration in the PET image can be improved.

It is needed to estimate an attenuation correction image (μ-map) in which the attenuation coefficient distribution inside the examination subject is subject to image to conduct the attenuation correction. As one example of the attenuation corrections, the corrected data are provided by eliminating an effect of γ-rays attenuation, wherein a transmittance of γ-rays is calculated by using the estimated attenuation coefficient image and the transmittance is divided from the measurement data of PET. In addition, as another example of attenuation correction, a corrected reconstruction image, in which the effect of γ-ray attenuation is eliminated by incorporating the estimated attenuation coefficient image into the calculating formula that is used for reconstructing the estimated attenuation coefficient image into the PET image, is provided.

Conventionally, it is listed as the first method that estimates the attenuation coefficient image irradiates the same nuclide radiation as the positron emission nuclide to the examination subject to obtain the transmission data and estimate the attenuation coefficient image using such transmission data. In addition, it is listed as the second method that estimates the attenuation coefficient image uses the CT-data obtained from an X-ray CT apparatus (CT; Computed Tomography) instead of the transmission data. According to such methods, an external radiation source, which irradiates the radiation to the examination subject, must be equipped with the PET apparatus.

Nowadays, a reconstruction algorithm has been proposed, by which neither the transmission data nor CT-data are needed (e.g., see non-patent documents 1, 2). According to the non-patent documents 1, 2, a distribution form of an attenuation coefficient sinogram can be estimated from the measurement data of PET that measures the detection time difference (called also as flight time difference, i.e., TOF: Time of Flight) information of the annihilation radiation (hereafter called as TOF-PET). Then, the PET image and an attenuation coefficient sinogram, or PET image and the attenuation coefficient image can be concurrently (simultaneously) estimated.

The reconstruction algorithm according to the non-patent documents 1, 2 estimates the data related to the PET image and the attenuation coefficient (e.g., attenuation coefficient sinogram) concurrently, so that it is also referred as a “concurrent (simultaneous) reconstruction algorithm”. Particularly, the concurrent reconstruction algorithm that estimates the PET image (radiation image) and the attenuation coefficient sinogram is referred as MLACF (Maximum Likelihood Attenuation Correction Factors) method, and the concurrent reconstruction algorithm that estimates the PET image and the attenuation coefficient image at the same time is refereed as MLAA (maximum likelihood attenuation and activity) method.

The transmission data or the CT-data to executes the attenuation correction processing of the PET image is no longer required because the PET image is generated using the concurrent reconstruction algorithm. Specifically, the external radiation source or the CT apparatus is no longer required to be installed to the PET apparatus, so that it can be avoided that the PET apparatus must be much bigger.

### RELATED PRIOR ART

### Non-Patent Document

Non-Patent Document 1: A. Rezaei, M. Defrise and J. Nuyts, “ML-reconstruction for TOF-PET with simultaneous estimation of the Attenuation Factors”, IEEE Trans. Med. Imag., 33 (7), 1563-1572, 2014.
Non-Patent Document 2: V. Y. Panin, H. Bal, M. Defrise, C. Hayden and M. E. Casey, “Transmission-less Brain TOF PET Imaging using MLACF”, 2013 IEEE Nuclear Science Symposium and Medical Imaging Conference, Seoul, Republic of Korea, 2013.

### ASPECTS AND SUMMARY OF THE INVENTION

### Objects to be Solved

Nevertheless, in the case of a conventional example having such structure, following problems remain to be solved.

As to the PET apparatus, there is the case in which the PET image is generated for a phantom (dummy) as an examination subject in addition to the case in which the living human is the examination subject. If the PET image is generated using the concurrent reconstruction algorithm in the case of the phantom used as the examination subject, the problem newly emerged that the degree of precision of the attenuation correction processing relative to the PET image become poor.

The inventors studied extensively the reason why the degree of precision of the attenuation correction worsens and found the following results. Specifically, referring to the left side figure of FIG. 9A, when the PET image L1 is generated for the living body S as the examination subject, the radio-active pharmaceutical diffuses into such as the capillary vessel right below the skin. Therefore, the range P1 (shaded area), into which the radioactive pharmaceutical diffuses, is the entire living body S including the body surface TH thereof. Specifically, the PET image L1, of the living body S, provides the radiation information for the entire living body S.

On the other hand, referring to the left side figure of FIG. 9B, when the PET image display element L2 is generated for the phantom F as the examination subject, the range P2 (shaded area) into which the radioactive pharmaceutical diffuses is not the entire area of the phantom F. The phantom F generally has an inner side injection area into which the radioactive pharmaceutical is injected, and a tubular housing N installed to surround such an inside injection area. The housing N is made of the material which blocks the radioactive pharmaceutical, and has some degree of thickness and hardness to support the strength of the phantom F. Therefore, the range P2, into which the radioactive pharmaceutical diffuses, is limited to the inner side injection area of the phantom F so that no radioactive pharmaceutical diffuses into the housing N.

And as long as the method of using the concurrent reconstruction algorithm, e.g., MLACF method or MLAA method is used, the range where the attenuation coefficient can be estimated is limited only to the range where a radioisotope is distributable, in principle. Specifically, the range where the attenuation coefficient image (μ-map) can be obtained is the range where the radioactive pharmaceuticals are distributable. In other words, when the living body S is the examination subject, the range Q1 in which μ-map can be obtained is the entire range of the living body S as shown in the middle figure of FIG. 9A. On the other hand, when the phantom F is the examination subject, the range Q2 where μ-map can be obtained is limited to the inner side injection element of the phantom F as shown in the middle figure of FIG. 9B and no attenuation coefficient can be estimated relative to the housing N of the phantom F.

In other words, given the living body S is the examination subject, the range Q1 where the μ-map can be actually obtained is the entire range of the living body S as shown in the right figure of FIG. 9A. Therefore, when the examination subject is the living body S, the attenuation correction processing with relatively high degree of precision become executable relative to the PET image L1.

On the other hand, given the examination subject is the phantom F, it is clear that the range of μ-map needed to adequately execute the attenuation correction relative to the PET image is different from the range where the μ-map can be actually obtained. When the examination subject is the phantom F, γ-rays emitted from the inner side injection area to the outside of the phantom F is partially absorbed by not only the structure of the inner side injection area but also the housing N. Particularly, the housing N is made of a relatively thick member, the attenuation level of the γ-rays is as high as unignorable. Accordingly, the μ-map R2 covering the entire area of the phantom F including the housing N is needed, as shown in the right figure of FIG. 9B, to execute the attenuation correction processing with the high level of precision relative to the PET image L2 of the phantom F.

However, the range Q2 is limited to the inner side injection area in which the μ-map is actually obtainable relative to the phantom F. Accordingly, even if the attenuation correction processing relative to the PET image L2 is executed using the μ-map actually obtained, the attenuation correction processing is executed while ignoring the attenuation coefficient in the housing N. As a result, if the PET image L2 of the phantom F is generated using the concurrent reconstruction algorithm, the problem becomes concerned that the degree of precision of the attenuation correction processing relative to the PET image L2 worsens.

The present invention has been developed while considering the above circumstances and the purpose of the present invention is to provide a diagnostic apparatus for nuclear medicine and a medicine and an estimation method of attenuation coefficient image that are capable of executing an attenuation correction processing with a high degree of precision relative to the radiation image without irradiating the examination subject using such as an external radiation source.

### Means for Solving the Problem

The present invention constitutes the following structure to achieve such a purpose.

Specifically, according the first aspect of the present invention, a diagnostic apparatus for nuclear medicine that generates a radiation image of a phantom using a radioactive pharmaceutical that emits a pair of annihilation radiations comprises: a memory element that stores a template radiation image, which is obtained in advance, denoting distribution of the radioactive pharmaceutical in the phantom and an entire attenuation coefficient image denoting distribution of the attenuation coefficient in the entire phantom; a detection ring that detects the pair of annihilation radiations; a data collection element that collects concurrent counting data based on information of the pair of annihilation radiations detected by the detector ring; a reconstruction element that generates an actual measurement radiation image by reconstructing the concurrent counting data; a position deviation calculation element that calculates difference between a position of the phantom when generating the actual measurement radiation image and a position of the phantom when obtaining the template radiation image as a position deviation level; a position correction element that generates a corrected attenuation coefficient image by correcting the position of the phantom incorporated in the entire attenuation coefficient image based on the position deviation level; and a first attenuation correction element that reconstructs the corrected radiation image, on which the attenuation correction is executed and denoting the distribution of the radioactive pharmaceutical in the phantom, by an attenuation correction processing using the correct attenuation coefficient image and the concurrent counting data.

In addition, according the second aspect of the present invention, an attenuation coefficient image estimation method of estimating an attenuation coefficient image of a phantom by the diagnostic apparatus for nuclear medicine comprises; a template data memory storing step of storing a template radiation image, which is obtained in advance, denoting distribution in the phantom of the radioactive pharmaceutical that emits a pair of annihilation radiations and an entire attenuation coefficient image denoting distribution of the attenuation coefficients in the entire phantom in a memory element, a detection step of detecting the pair of annihilation radiations emitted from the inside of the phantom using detection rings which are in place in the positions surrounding the phantom; a data collection step of collecting concurrent counting data based on the information of the pair of annihilation radiations detected in the detection step; a reconstruction step of generating an actual measurement radiation image by reconstructing the concurrent counting data; a position deviation calculation step of calculating difference between a position of the phantom when obtaining an actual measurement radiation image and a position of the phantom when obtaining the template radiation image as a position deviation level; a position correction step of estimating a corrected attenuation coefficient image by correcting the position of the phantom incorporated in the entire attenuation coefficient image based on the position deviation level that is calculated in the position deviation calculation step.

### Effects of the Present Invention

According to the first aspect of the present invention, the template radiation image and the entire attenuation coefficient image are obtained in advance, and such two images are stored in the memory storage in advance. And the estimation of attenuation coefficient image is executed using the template radiation image and the entire attenuation coefficient image, which are stored in advance, every time when the radiation image as to the phantom is generated, Specifically, the concurrent counting data relative to the phantom subjected to the examination are obtained, and the actual measurement radiation image is generated by reconstructing the concurrent counting data. And the position deviation level between the actual measurement radiation image and the template radiation image is calculated, and then the entire attenuation coefficient image, which is obtained in advance, is corrected based on such a position deviation level. The entire attenuation coefficient image is corrected, so that the corrected attenuation coefficient image used in the attenuation correction processing relative to the actual measurement radiation image can be generated.

The position of distribution of the attenuation coefficient relative to the corrected attenuation coefficient image is corrected so as to correspond to the position of the phantom image incorporated in the actual measurement radiation image by the position correction element. Therefore, the attenuation correction element becomes capable of executing the attenuation correction processing by the corrected attenuation coefficient image as for the concurrent counting data collected when generating the actual measurement radiation image.

The entire attenuation coefficient image is the image wherein the attenuation coefficient in the entire phantom is estimated in advance. Accordingly, the corrected attenuation coefficient image for the actual measurement radiation image, which is obtained by correcting the position of the phantom incorporated in the entire attenuation coefficient image, is also the image of which the attenuation coefficient is estimated relative to the entire phantom. Therefore, the first attenuation correction element executes the attenuation correction processing for the concurrent counting data using the corrected attenuation coefficient image, so that the attenuation correction is executed relative to the entire phantom and the corrected radiation image can be obtained as the image denoting the distribution of the radioactive pharmaceutical in the phantom.

There is no need to irradiate the radiation to the phantom which is the examination subject in the step of estimating the corrected attenuation coefficient image. Accordingly, the corrected radiation image of the phantom, for which the attenuation correction processing is executed, is obtained with a high degree of precision without installing the external radiation source to the diagnostic apparatus for nuclear medicine. Specifically, the degree of precision of the attenuation correction processing relative to the concurrent counting data can be improved in the non-clinical evaluation using the phantom while avoiding that the diagnostic apparatus for nuclear medicine is needed to be made bigger.

According to the second aspect of the present invention, the template radiation image and the entire attenuation coefficient image are stored in the memory during the template data storing step. And the concurrent counting data based on the pair of annihilation radiations emitted from the inside phantom is collected during the detection step and the data collection step. In the reconstructing step, the actual measurement radiation image is generated by reconstructing the concurrent counting data. In the position deviation calculation step, a difference between the position of the phantom when obtaining the actual measurement radiation image when obtaining the template radiation image is calculated as a position deviation level. In the position correction step, the corrected attenuation coefficient image is estimated by correcting the position of the entire attenuation coefficient image based on the position deviation level.

The template radiation image is the image denoting the distribution of the radiation in the phantom and the entire attenuation coefficient image is the image wherein the distribution of the attenuation coefficient is estimated relative to the entire phantom. Therefore, the corrected attenuation coefficient image, which is obtained by correcting the position of the phantom incorporated in the entire attenuation coefficient image, is also the image wherein the distribution of the attenuation coefficient is estimated in the entire phantom. Therefore, the attenuation correction processing is executed relative to the entire area of the phantom by using the corrected attenuation coefficient image.

And in a position correction step, the corrected attenuation coefficient image is estimated by correcting the position of the phantom incorporated in the entire attenuation coefficient image according to the position deviation level between the template radiation image and the actual measurement radiation image. Specifically, the position of distribution of the attenuation coefficient in the corrected attenuation coefficient image HF is corrected so as to correspond to the position of the phantom image incorporated in the actual measurement radiation image. Therefore, the attenuation correction processing relative to the concurrent counting data collected when generating the actual measurement radiation image can be executed by the corrected attenuation coefficient image. Accordingly, the corrected attenuation coefficient image, to which the attenuation correction processing for the concurrent counting data of the phantom is applicable, can be estimated without using the external radiation source. Specifically, the degree of precision of the attenuation correction processing relative to the concurrent counting data can be improved in the non-clinical evaluation using the phantom while avoiding that the diagnostic apparatus for nuclear medicine is needed to be made bigger.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a perspective view and block diagram illustrating a schematic structure of a PET apparatus according to the Embodiment.

FIG. 2 is a perspective view illustrating a schematic structure of a radiation detector according to the Embodiment.

FIG. 3 is a functional block diagram illustrating the PET apparatus according to the Embodiment.

FIG. 4A is a schematic perspective view illustrating a structure of the phantom according to the Embodiment.

FIG. 4B is a plan view of the phantom according to the Embodiment.

FIG. 5 is a flow chart illustrating an operation of the PET apparatus according to the Embodiment.

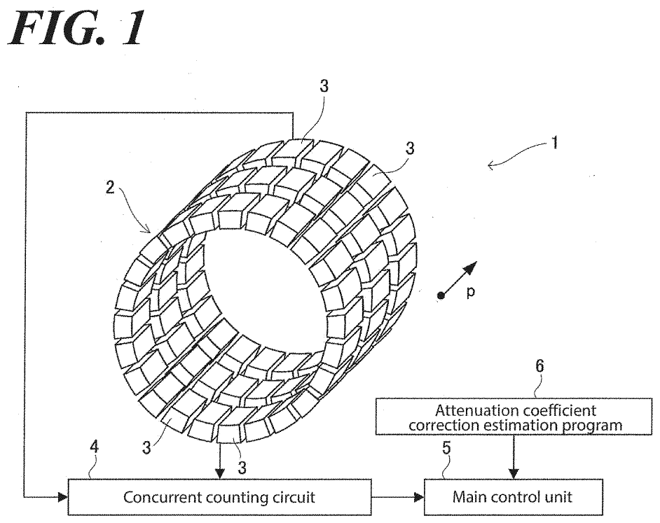

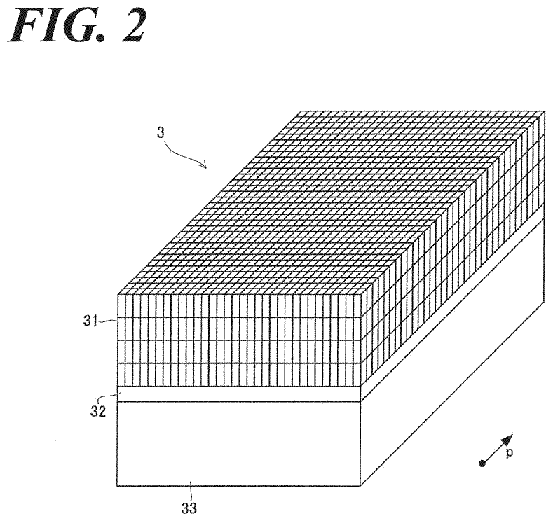

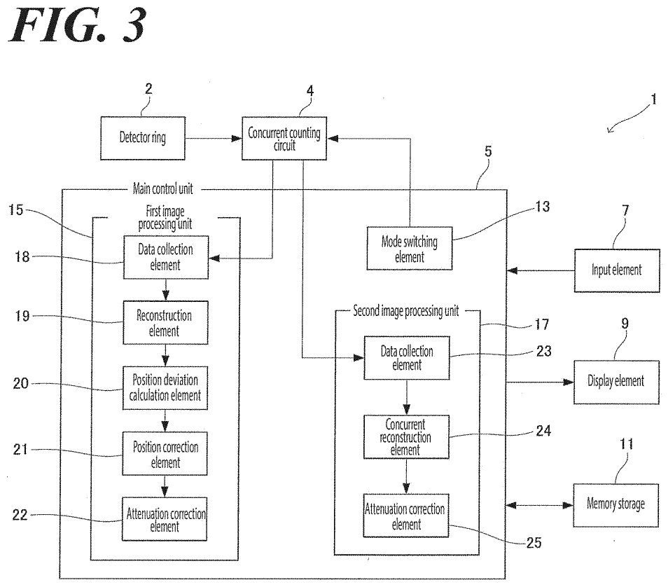

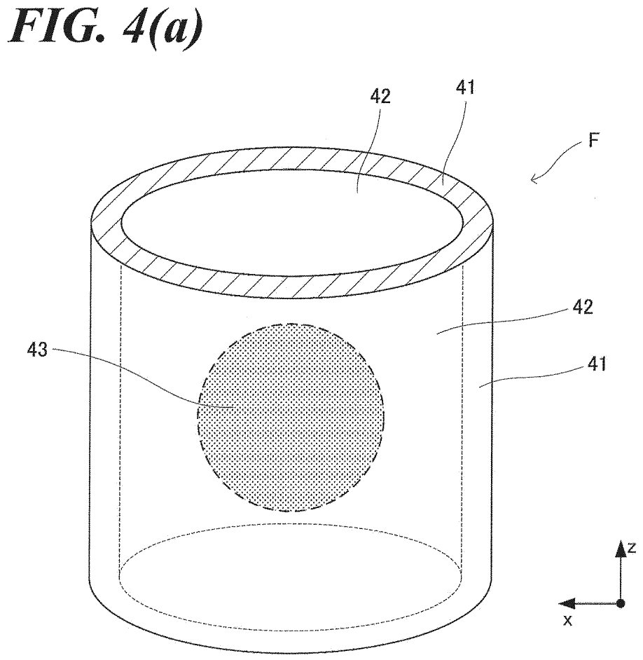

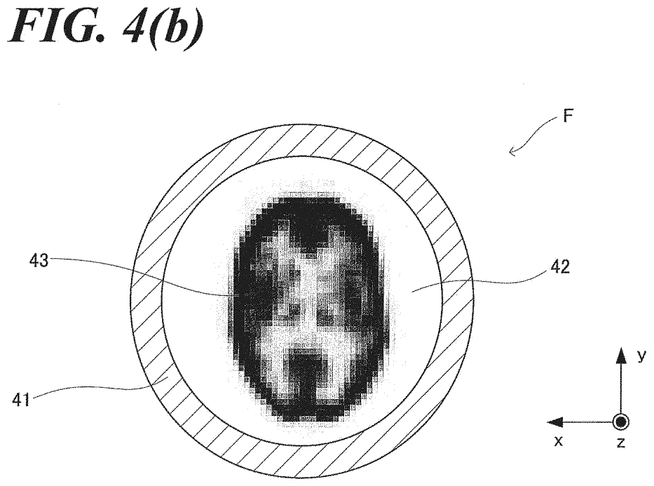

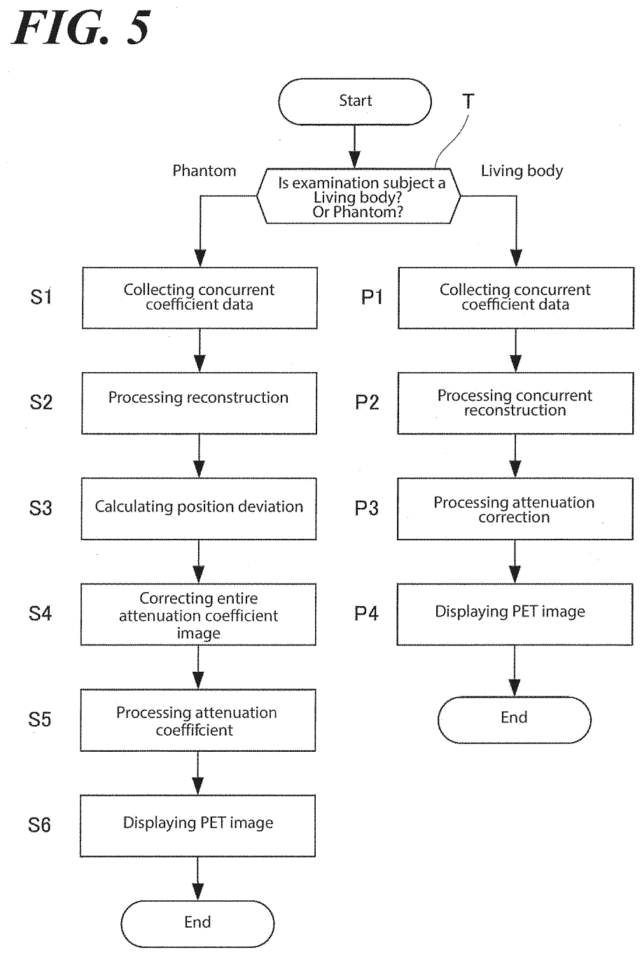

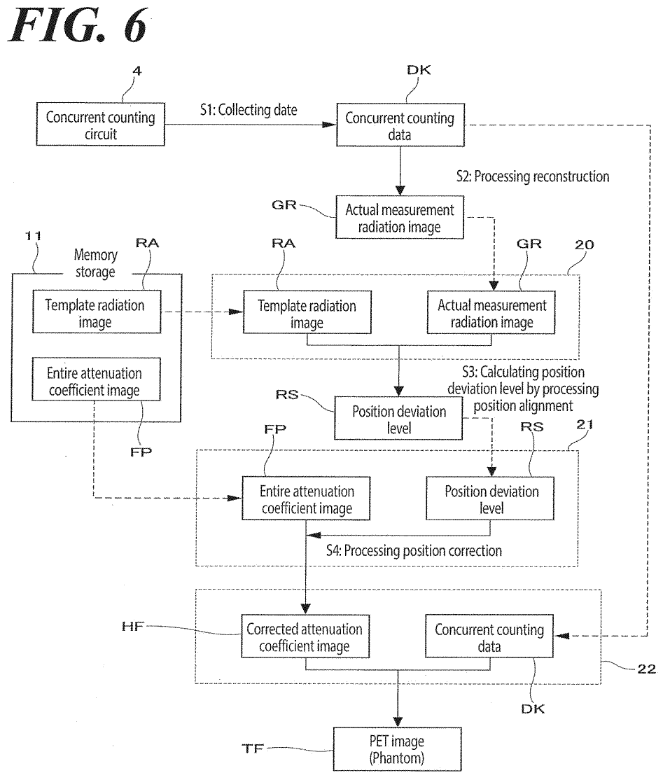

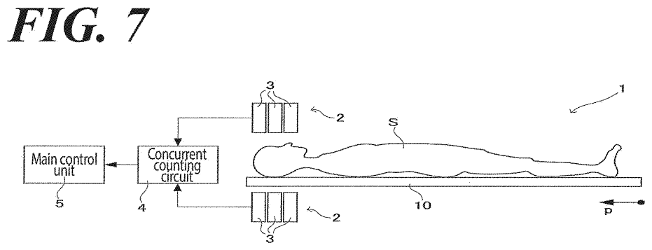

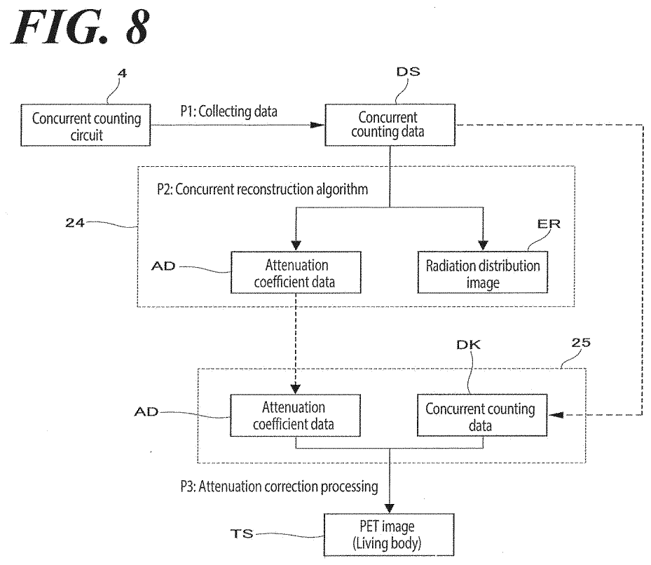

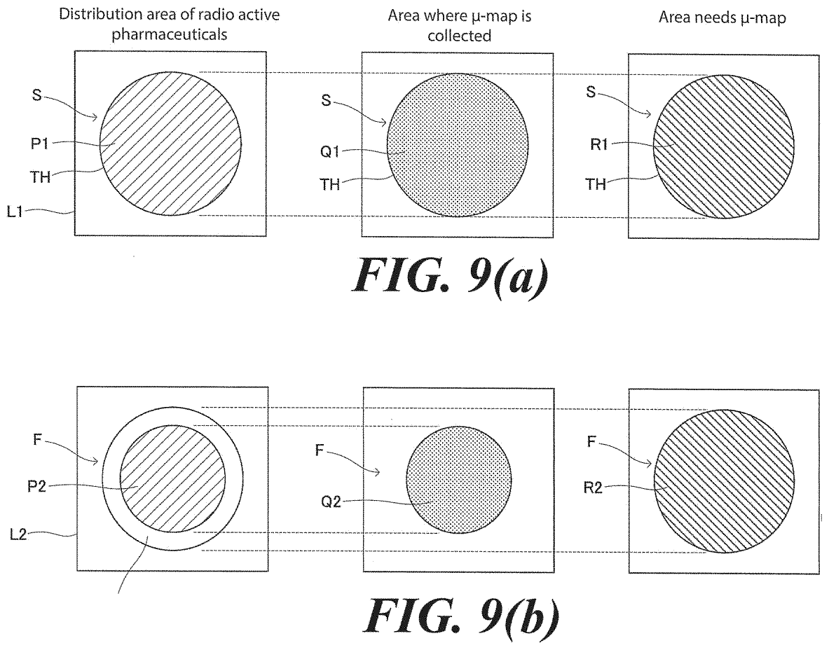

FIG. 6 is a processing procedure for the first mode that estimates an attenuation coefficient image of the phantom and a data flow using the PET apparatus according to the Embodiment.

FIG. 7 is a longitudinal section view illustrating the PET apparatus in the state of the second mode according to the Embodiment.

FIG. 8 is a processing procedure for the second mode that estimates an attenuation coefficient image of a living body and a data flow using the PET apparatus according to the aspect of the Embodiment.

FIG. 9A is a diagram illustrating problems due to a conventional configuration given the examination subject is a living body, wherein the left t figure denotes the area where the radioactive pharmaceutical can be distributes, the middle figure indicates the area where μ-map can be actually obtained, and the right figure denotes the area where μ-map is needed to execute the ideal attenuation correction.

FIG. 9B is a diagram illustrating problems due to a conventional configuration given the examination subject is a phantom, wherein the left figure indicates the area where the radioactive pharmaceutical can be distributed, the middle figure denotes the area where μ-map can be actually obtained, and the right figure denotes the area where a μ-map is needed to execute the ideal attenuation correction.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

Referring to Figures, the inventor sets forth the Embodiment of the present invention.

(Illustration of the Entire Configuration)

According to the Embodiment referring to FIG. 1, the PET apparatus 1 comprises a detection ring 2 which is layered and arranged in the p-direction. The detection ring 2 is in place as surrounding the vicinity of the examination subject. As an example, the p-direction corresponds to the body axis direction of the examination subject. A plurality of γ-ray detectors 3 is embedded in the detection ring 2. The γ-ray detector 3 corresponds to the radiation detector of the present invention.

In addition, the PET apparatus 1 comprises a concurrent counting circuit 4 and a main control unit 5. Referring to FIG. 1, only two connecting wires from the γ-ray detector 3 to the concurrent counting circuit 4 are shown, but in fact, the number of concurrent counting circuits 4 is the same as the total channel number of the photomultipliers 33 (refer to FIG. 2) that the γ-ray detector 3 comprises. A main control unit 5 is a processor comprising information processing means such as a central processing unit (CPU), which executes a variety of arithmetic processing. As an example of a variety of arithmetic processing, the main control unit 5 executes the attenuation coefficient image estimation program 6 for the attenuation coefficient image estimation processing.

Referring to FIG. 2, the γ-ray detector 3 comprises a scintillator block 31, a light guide 32 and a photomultiplier 33. The scintillator block 31 comprises scintillator elements, which convert radiation to light, are three dimensionally arranged. Specifically, according to the present embodiment, the γ-ray detector 3 is a DOI detector comprising a plurality of layers in the depth direction. Referring to FIG. 2, the DOI detector having four layers are illustrated but the number of layers are not particularly limited thereto. The DOI detector is formed by layering the respective scintillator elements in the depth direction of radiation and the coordinate data of the depth (DOI: Depth of Interaction) direction, in which an interaction takes place, and the traverse direction (parallel direction to the incident plan) is obtained by the centroid operation.

The respective scintillator elements forming the scintillator block 31 detect γ-rays by converting γ-rays to lights. The light guide 32 is optically connected with the scintillator block 31. The photomultiplier 33 is optically connected with the light guide 32 and converts light to an electric signal.

Specifically, when the γ-ray is incident into the scintillator block 31, the scintillator element detects γ-ray and then emits light. Lights emitted from the scintillator element are fully diffused by the scintillator block 31 and then become incident into the photomultiplier 33 via the light guide 32. The photomultiplier 33 converts lights multiplied by the scintillator block 31 to electric signals. The converted electric signals are sent to the concurrent counting circuit 4 as pixel values.

The concurrent counting circuit 4 detects the concurrent counting data from the γ-ray information detected by the detection ring 2. In particular, when the radioactive pharmaceutical is administered into the examination subject e.g., such as the living body or the phantom, two γ-rays are emitted by annihilation of positrons of positron emission type RI. The concurrent counting circuit 4 checks the position of the scintillator block 31 and the incident timing of the γ-rays and then decides that the input electric signals are adequate only when the γ-rays are incident in the two scintillator blocks 31, which are arranged at both sided of the examination subject, within a certain period of time. When the γ-rays are incident only into only one scintillator block 31, the concurrent counting circuit 4 discards such an incident. The electric signals decided as adequate data by the concurrent counting circuit 4 are sent as the concurrent counting data (emission data) from the concurrent counting circuit 4 to the main control unit 5. The main control unit 5 conducts a variety of operations using such as the concurrent counting data and generates the PET image denoting distribution of the radioactive pharmaceuticals in the examination subject.

Referring to FIG. 3, the PET apparatus 1 further comprises an input element 7, a display element 9 a memory storage 11. The input element 7 inputs an operation instruction given by an operator. For example, the input element 7 may include sch as a keyboard input device, a touch panel input device and a mouse input device. The display element 9 displays a variety of data. e.g., image information, and may include such as a liquid crystal display. The PET image generated by the main control unit 5 is displayed on the display element 9.

The memory storage 11 stores a variety of data, e.g., the attenuation coefficient image estimation program 6, the concurrent counting data and a variety of image information. An example of the memory storage 11 is a non-volatile memory. In addition, the memory storage 11 preliminarily stores the template radiation image RA and the entire attenuation coefficient image FP as described later.

Referring to FIG. 3, the main control unit 5 comprises a mode switching element 13, a first image processing unit 15 and a second image processing unit 17. The mode switching element 13 switches the first mode that generates the PET image using the first image processing unit 15 and the second mode that generates the PET image using the second image processing unit 17 in accordance with the examination subject of the PET apparatus 1. According to the present Embodiment, when the examination subject is the phantom F, the first mode is selected and when the examination subject is the living body S, the second mode is selected. For example, the mode switching element 13 switches the first mode and the second mode when an operator operates the mode switching switch installed to the input element 7.

The first image processing unit 15 that conducts a variety of image processings to generate the PET image of the phantom TF comprises a data collection element 18, a reconstruction element 19, a position deviation calculation element 20, a position correction element 21 and an attenuation correction element 22. The data collection element 18 collects the concurrent counting data sent from the concurrent counting circuit 4. The reconstruction element 19 conducts the reconstruction processing for the concurrent counting data, which the data collection element 18 collects, to generate the actual measurement radiation image GR of the phantom F.

The position deviation calculation element 20 compares the actual measurement radiation image GR generated by the reconstruction element 19 and the template radiation image RA to calculate the difference between the positions of the phantom F respectively incorporated therein as the position deviation level RS. The position correction element 21 corrects the entire attenuation coefficient image FP stored in the memory storage 11 based on the information of the position deviation level RS. The corrected attenuation coefficient image HF is generated by correction that the position correction element 21 executes.

The attenuation correction element 22 executes the attenuation correction processing relative to the concurrent counting data, which the data collection element 18 collects, by using the corrected attenuation coefficient image HF. The PET image TF of the phantom F is reconstructed by the attenuation correction processing that the attenuation correction element 22 executes. The PET image TF, on which the attenuation correction is executed, denotes distribution of the radioactive pharmaceuticals in the phantom F. The attenuation correction element 22 corresponds to the first attenuation correction element according to the aspect of the present Embodiment. The PET image TF corresponds to the corrected radiation image of the aspect of the present Embodiment.

The second image processing unit 17 that executes a variety of image processings to generate the PET image TS of the living body S comprises a data collection element 23, a concurrent reconstruction element 24 and the attenuation correction element 25. The data collection element 23 collects the concurrent counting data sent from the concurrent counting circuit 4. The concurrent reconstruction element 24 generates the attenuation coefficient data AD and the radiation distribution image ER by reconstructing the concurrent counting data using the concurrent reconstruction algorithm.

The attenuation correction element 25 executes the attenuation correction processing relative to the concurrent counting data, which the data collection element 23 collects, by using the attenuation coefficient data AD. The PET image TS of the living body S is reconstructed by the attenuation correction processing that the attenuation correction element 25 executes. The PET image TS, on which the attenuation correction is executed, denotes distribution of the radioactive pharmaceuticals in the living body S. In the PET image TS corresponds to the corrected image according to the aspect of the present Embodiment. The attenuation correction element 25 corresponds to the second attenuation correction element according to the present Embodiment.

<Structure of the Phantom>

Referring to FIG. 4A, 4B, the inventors set forth the structure of the phantom F used for the PET apparatus 1. FIG. 4A is the perspective view of the phantom F and FIG. 4B is a plan view of the phantom F. The phantom F comprises a housing 41, the inner side storage element 42 and a living body dummy 43. The housing 41 forms the external wall of the phantom F and is a bottomed hollow cylinder overall. According to the present embodiment, the housing 41 is the hollow cylindrical member but the shape of the housing 41 can be arbitrarily modified. It is preferable that the housing 41 has thickness and hardness of more than a predetermined value to ensure the strength of the phantom F. A material forming the housing 41 may be e.g., an acrylic resin.

The inner side storage element 42 corresponds to the hollow part formed inside the housing 41, the living body dummy 43 is stored in the inner side storage element 42. The living body dummy 43 is the model of the part or whole of the living body e.g., a living human or an animal and has a hollow portion is formed in the inside thereof. When the radioactive pharmaceutical is injected in the hollow portion, the γ-rays are emitted from the living body dummy 43. According to the present embodiment, the living body dummy 43 is a model of a human brain.

<Operation of PET Apparatus>

The inventors set forth the operation of the PET apparatus according to the embodiment. FIG. 5 is a flow chart illustrating a series of operations to obtain PET image of the examination subject (image target) using the PET apparatus 1.

When the PET apparatus according to the embodiment is used, the template radiation image RA and the entire attenuation coefficient image FP are preliminarily stored in the memory storage 11. In addition, the programs used for processing a variety of operations are preliminarily stored in the memory storage 11. The step of storing preliminarily the template radiation image RA and the entire attenuation coefficient image FP in the memory storage 11 corresponds to the temple data storing processing of the aspect of the present embodiment.

The template radiation image RA is the image denoting distribution of the radioactive pharmaceuticals in the phantom F. The template radiation image RA is obtained by image preliminarily the radiation image relative to the phantom F.

The entire attenuation coefficient image FP is the image denoting distribution of the attenuation coefficient of the entire phantom F. In addition, the entire attenuation coefficient image FP is the image that enables the attenuation correction processing relative to the phantom image of the template radiation image RA. Specifically, the attenuation correction processing can be executed relative to the emission data of the phantom F by using the entire attenuation coefficient image FP, which is obtained when the template radiation image RA is generated in the past operation.

The entire attenuation coefficient image FP is the image that enables the attenuation correction processing relative to the template radiation image RA. Specifically, the entire attenuation coefficient image FP enables the attenuation correction processing relative to the concurrent counting data collected when generating the template radiation image RA. In addition, the relationship between the position of the phantom F in the entire attenuation coefficient image FP and the position of the phantom F in the template radiation image RA is already known. According to the present Embodiment, the template radiation image RA and the entire attenuation coefficient image FP are defined as the image that is generated relative to the phantom F arranged at the same position.

An example of the method of obtaining preliminarily the entire attenuation coefficient image FP is the method of conducting CT image for the phantom F as the target using a PET/CT apparatus and then estimating the attenuation coefficient using the data of the obtained CT image.

In addition, an example of obtaining preliminarily the entire attenuation coefficient image FP is the method of calculating distribution of the attenuation coefficient relative to the entire phantom F by a geometric operation. Specifically, each of the housing 41, the inner side housing element 42 and the living body dummy 43 forms the phantom F has preliminarily the information such as the position that is in place in the phantom and the material that is used for forming therein. The attenuation of the γ-rays can be preliminarily calculated provided the construction material is already known, so that the distribution of the attenuation coefficient in the entire phantom F can be preliminarily calculated by the geometric operation method by knowing preliminarily the type of construction material and the position thereof.

When the entire attenuation coefficient image FP is obtained by the geometric operation, the entire attenuation coefficient image FP can be preliminarily obtained without calculating the attenuation coefficient by actually irradiating the radiation to the phantom F. Specifically, it is advantageous that another radiation image apparatus separated from the PET apparatus, e.g., the X-ray CT apparatus or the PET/CT apparatus, is not needed when the entire attenuation coefficient image FP is generated.

Step T (Section of a Mode)

First an operation mode is selected when the PET image is generated using the PET apparatus 1. Specifically, as denoted by the sign T in FIG. 5, the mode is switched to divaricate the operation of the PET apparatus 1 depending on whether the examination subject is the phantom or the living body. When the examination subject is the phantom, the operator switches the operation mode of the PET apparatus 1 to the first mode by operating the mode changing switch, not shown in FIG. When switched to the first mode, the PET apparatus 1 generates the PET image of the phantom according to each step of Step 1 or Step S6.

On the other hand, when the examination subject is a living body e.g., the living human or an animal, the operator switches the operation mode of the PET apparatus 1 to the second mode by operating the mode changing switch. When switched to the second mode, the PET apparatus 1 generates the PET image of the living body according to each step of Step P1 or Step P4.

Here, the inventors first set forth the step of Step S1 or Step S6 relative to the first mode. Referring to FIG. 6, the flow of a variety of data and a variety of operations in the first mode used to obtain the PET image are illustrated, wherein the examination subject is the phantom.

Step S1 (Collection of the Concurrent Counting Data)

When the PET image is generated for the phantom as the examination subject, the radioactive pharmaceutical labeled with the positron emission nuclide is injected into the living body dummy 43 housed inside the phantom F. And the phantom F, into which the radioactive pharmaceutical is injected, is in place in the center of the detection ring 2. And the operator inputs an instruction to select the first mode by operating such as the mode switching switch installed to the input element 7.

Once the radioactive pharmaceutical is injected into the living body dummy 43, a pair of γ-rays emits along with annihilation of the positron. The pair of the γ-rays emitted thereby travels in the opposite direction each other and is incident into the scintillator block 31 through the inner side storage element 42 and the housing 41. The concurrent counting circuit 4 detects the position and the timing at which the γ-rays are incident in the respective scintillator blocks 31. And when the pair of the γ-rays are incident into the scintillator block 31 within the specific period of time, the concurrent counting circuit 4 sends the data of the position and the timing at which the respective γ-rays are incident to the main control unit 5 as the concurrent counting data DK.

When the switching operation for switching to the first mode, the signal carrying the selection of the first mode is sent to the mode switching element 13. In such a case, the mode switching element 13 controls the concurrent counting circuit 4 so that the concurrent counting data detected by the concurrent counting circuit 4 is sent to the first image processing unit 15 in the main control unit 5. Specifically, when the first mode is selected, the detection signal of the γ-rays that the concurrent counting circuit 4 decides as the concurrent counting data DK is sent to the data collection element 18 that the first image processing unit 15 comprises.

The data collection element 18 collects the respective concurrent counting data DK decided by the concurrent counting circuit 4. The concurrent counting circuit 4 sends sequentially the concurrent counting data DK to the data collection element 18 following an emission of γ-rays, so that a number of concurrent counting data DK are being accumulated. The format of the concurrent counting data DK may be a list mode format or a sinogram format. The concurrent counting data DK collected by the data collection element 18 are sent to the reconstruction element 19. In addition, the concurrent counting data DK are sent to the memory storage 11 to be stored. The step of Step S1 corresponds to the data detection processing and the data collection processing of the present embodiment.

Step S2 (Reconstruction Processing)

The reconstruction element 19 executes the reconstruction processing for the concurrent counting data DK collected by the data collection element 18 to generate the actual measurement radiation image GR. The actual measurement radiation image GR is the image denoting distribution of the radioactive pharmaceuticals in the phantom F (i.e., phantom F as the examination subject) which is in place inside the detector rings 2. Specifically, the γ-ray emission position, i.e., the position of the radioactive pharmaceutical is specified by the straight line (LOR: Line of Response) connecting two scintillator blocks 31 that detect a pair of γ-rays and the difference between detection times of a pair of γ-rays. Therefore, the image denoting distribution of the radioactive pharmaceuticals can be generated by the operational processing that executes the reconstruction for the concurrent counting data DK. However, the actual measurement radiation image GR is the radiation image on which the attenuation correction processing is not executed, so that in such a point, it is the different image from the PET image TF as described later.

The technical method used for the reconstruction processing of the actual measurement radiation image GR may be arbitrarily selected, and may be an analytical reconstruction technical method e.g., such as the FRP method (FBP: Filtered Back Projection). In addition, the successive approximation reconstruction method, for example, OESM method or MLEM method, may be applied (OSEM: Ordered Subset Expectation Maximization, MLEM: Maximum likelihood Expectation Maximization). In addition, an example of the noise reduction processing in the reconstruction processing may be the Gaussian filter or the non-local means filter. In addition, the noise reduction processing may be any one of a pretreatment applied for the projection data, a processing which is executed in the reconstruction algorithm, and a postprocessing which is executed after generating the reconstruction image. The actual measurement radiation image GR of the phantom F, which is generated by the reconstruction processing, is sent from the reconstruction element 19 to the position deviation calculation element 20. The step of Step S2 corresponds to the reconstructing step according to the aspect of the present embodiment.

Step S3 (Calculation of the Position Deviation Level)

Once the data of the actual measurement radiation image GR are sent to the position deviation calculation element 20, the position deviation calculation element 20 reads out the template radiation image RA preliminarily stored in the memory storage 11 referring to FIG. 6. And the position deviation calculation element 20 calculates the position deviation level RS of the phantom F using the template radiation image RA and the actual measurement radiation image GR. Specifically, the position deviation calculation element 20 executes the image operation processing (position alignment processing) so that the position of the phantom F incorporated in the template radiation image RA coincides with the position of the phantom F incorporated in the actual measurement radiation image GR. And a parameter relative to the difference between the position of the phantom F incorporated in the template radiation image RA, which is obtained by the position alignment processing of the phantom F, and the position of the phantom F incorporated in the actual measurement radiation image GR is calculated as a position deviation level RS.

The phantom that is used to take the template radiation image RA in advance and the phantom that is used to take the actual measurement radiation image GR at the present time have the same structure in general. Therefore, the position deviation calculation element 20 can calculate the position deviation level RS by executing the image processing so that the position of the phantom incorporated in the template radiation image RA and the position of the phantom incorporated in the actual measurement radiation image GR coincide.

The position alignment processing that the position deviation calculation element 20 executes can be conducted arbitrarily using any technical method of aligning the position relative to a three-dimensional image. The position alignment processing may be an operation processing using e.g., Nelder-Mead method, a downhill simplex method, or amoeba method alignment processing.

The parameter included in the position deviation level RS may be e.g., distance parameters that move respective three axes orthogonal each other in parallel when the positions of respective phantoms F are aligned. In addition, the parameter included in the position deviation level RS may be e.g., angle parameters that move the respective orthogonal three axes around the respective axes in rotation when the positions of respective phantoms F are aligned. Further, in Step S3, the timing when the position deviation calculation element 20 reads out the template radiation image RA may be arbitrarily changed independently from the timing when the actual measurement radiation image GR is sent to the position deviation calculation element 20. The data of the position deviation level RS are sent from the position deviation calculation element 20 to the position correction element 21. Step S3 corresponds to the position deviation calculation step according to the aspect of the present Embodiment.

Step S4 (Correction of the Entire Attenuation Coefficient Image)

Once the data of the position deviation level RS are sent to the position correction element 21, the position correction element 21 reads out the entire attenuation coefficient image FP stored preliminarily in the memory storage 11 as shown in FIG. 6. And the position correction element 21 corrects the entire attenuation coefficient image FP based on the position deviation level RS. Specifically, the entire image of the phantom F incorporated in the entire attenuation coefficient image FP changes the position thereof following such as the parallel moving processing and the rotation moving processing in accordance with the position deviation level RS by the correction processing that the position correction element 21 executes. The position correction element 21 corrects the entire attenuation coefficient image FP so that the corrected attenuation coefficient image HF is newly generated.

The entire image of the phantom F incorporated in the entire attenuation coefficient image FP obtained in past time and the entire image of the phantom F in the template radiation image RA coincide in the position each other. On the other hand, the entire image of the phantom F incorporated in the entire attenuation coefficient image FP obtained in past time and the entire image of the phantom F incorporated in the actual image GR obtained at present do not coincide in the position. Then the position correction element 21 corrects the position of the phantom F incorporated in the entire attenuation coefficient image FP based on the position deviation level RS and obtains (estimates) newly the corrected attenuation coefficient image HF.

The position deviation level RS corresponds to the difference between the position of the entire image of the phantom F incorporated in the template radiation image RA and the position of the entire image of the phantom F incorporated in the actual measurement radiation image GR. Therefore, the entire image of the phantom F incorporated in corrected attenuation coefficient image HF and the entire image of the phantom F incorporated in the actual measurement radiation image GR would coincide in the position each other. In other words, whereas the position of the phantom F incorporated in the entire attenuation coefficient image FP is the position where the phantom F was placed when obtaining the template radiation image RA in past time, the entire image of the phantom F incorporated in the corrected attenuation coefficient image HF is corrected so as to become the position corresponding the position where the phantom F is placed when obtaining the concurrent counting data DK and the actual measurement radiation image GR at present. The data of the estimated and corrected attenuation coefficient image HF are sent from the position correction element 21 to the attenuation correction element 22. Step S4 corresponds to the step of the position correction step according to the aspect of the present Embodiment.

Step S5 (Attenuation Correction Processing)

Once the data of the corrected attenuation coefficient image HF is sent to the attenuation correction element 22, the attenuation correction element 22 reads out the concurrent counting data DK from the memory storage 11, referring to FIG. 6. In addition, the attenuation correction element 22 may directly send the concurrent counting data DK from the data collection element 18. And the attenuation correction element 22 executes the attenuation correction processing for the concurrent counting data DK using the corrected attenuation coefficient image HF. The PET image TF of the phantom F is reconstructed by the attenuation correction processing that the attenuation correction element 22 executes. The PET image TF is the image that denotes distribution of the radioactive pharmaceuticals in the phantom F which is the examination subject and in addition, the instant image on which the attenuation correction is executed relative to the entire phantom F.

As one example of the attenuation correction processing that the attenuation correction element 22 executes, the corrected concurrent counting data DK is reconstructed to generate the PET image TF following the transmittance of the γ-ray is calculated from the estimated corrected attenuation coefficient image HF and the correction, in which the γ-ray transmittance is divided from the concurrent counting data DK, is executed. In addition, as another example of the attenuation correction processing that the attenuation correction element 22 executes, the correction to provide the reconstruction image (PET image TF) is executed, wherein an effect of γ-ray attenuation is eliminated by incorporating the estimated corrected attenuation coefficient image HF into the calculating express that is used for reconstructing the concurrent counting data DK.

The position of the phantom F incorporated in the corrected attenuation coefficient image HF is corrected by the position correction element 21 so as to correspond to the arrangement of the phantom F at the time when the concurrent counting data DK was obtained in past time. Therefore, the corrected attenuation coefficient image HF is the image capable of processing the attenuation correction by applying to the concurrent counting data DK obtained at present time. Accordingly, the PET image TF can be reconstructed from the concurrent counting data DK obtained relative to the phantom F mounted to the PET apparatus 1 by using the estimated and corrected attenuation coefficient image HF.

Step S6 (Display of the PET Image)

The data of the PET image TF are sent to the display element 9 and the memory storage 11. The display element 9 displays the PET image and the memory storage 11 stores the data of the PET image TF. The PET image TF is displayed on the display element 9, so that the operator can visually recognize distribution of the radioactive pharmaceuticals in the phantom F. The operator conducts the non-clinical evaluation as to capability of the attenuation correction processing and the processing capability of the PET image in the PET apparatus 1 using the data of the PET image TF. The imaging processing of the PET image is completed by Step S1 or Step S6 processing when the phantom F is specified as the examination subject.

Next the inventors set forth the step of Step P1 or Step P4 according to the second mode. Referring to FIG. 8, the flow of a variety of data and a variety of processings in the second mode used to obtain the PET image are illustrated, wherein the examination subject is the living body S.

Step P1 (Collection of the Concurrent Counting Data)

When the PET image is generated for the living body S as the examination subject, the radioactive pharmaceutical labeled with the positron emission nuclide is administered into the living body S. And referring to FIG. 7, the living body S into which the radioactive pharmaceutical is administered is loaded on the table 10 and the examination region (head in the present Embodiment) is loaded at the position where the detector rings 2 are surrounding. And the operator inputs an instruction to select the second mode by operating such as the mode switching switch installed to the input element 7.

A pair of the γ-rays emitted by the annihilation of the positron travels in the opposite direction each other and is incident into the scintillator block 31. The concurrent counting circuit 4 detects the position and the timing at which the γ-rays are incident in the respective scintillator blocks 31. And the pair of the γ-rays incident into the scintillator block 31 within the specific period of time, the concurrent counting circuit 4 sends the data of the position and the timing at which the respective γ-rays are incident into the main control unit 5 as the concurrent counting data. In addition, the concurrent counting data obtained relative to the living body S is denoted by the sign DS, so that the concurrent counting data DK relative to the phantom F can be distinguished therefrom.

When the switching operation for switching to the second mode, the signal carrying the selection of the second mode is sent to the mode switching element 13. In such a case, the mode switching element 13 controls the concurrent counting circuit 4 so that the concurrent counting data DS detected by the concurrent counting circuit 4 are sent to the second image processing unit 17 in the main control unit 5. Specifically, when the second mode is selected, the detection signal of the γ-rays that the concurrent counting circuit 4 decides as the concurrent counting data DS is sent to the data collection element 23 that the second image processing unit 17 comprises.

The data collection element 23 collects the respective concurrent counting data DS decided by the concurrent counting circuit 4. The concurrent counting circuit 4 sends sequentially the concurrent counting data DS to the data collection element 23 following an emission of γ-rays, so that a number of concurrent counting data DS are being accumulated in the data collection element 23. The concurrent counting data DS collected by the data collection element 23 are sent to the concurrent reconstruction element 24. In addition, the concurrent counting data DS are sent to the memory storage 11 to be stored.

Step S2 (Concurrent Reconstruction Processing)

The concurrent reconstruction element 24 executes the concurrent reconstruction processing for a number of the concurrent counting data DS collected by the data collection element 23. Specifically, the concurrent reconstruction element 24 executes the concurrent reconstruction processing using the concurrent reconstruction algorithm and the radiation distribution image ER and the attenuation coefficient data AD are generated thereby. The radiation distribution image ER is the image that denotes distribution of the radioactive pharmaceuticals in the living body S, and on which no attenuation correction processing is executed. The attenuation coefficient data AD is the data denoting the distribution of the attenuation coefficient in the entire of the living body S, and one example thereof is the attenuation coefficient sinogram or the attenuation coefficient image.

The concurrent reconstruction algorithm that the concurrent reconstruction element 24 executes may be an arbitrary algorithm. Examples of the concurrent reconstruction algorithm may include the algorithm that executes the concurrent reconstruction processing using MLACF method, and the algorithm that executes the concurrent reconstruction processing using MLAA method. When the concurrent reconstruction element 24 uses the concurrent reconstruction algorithm for MLACF method, the data of the attenuation coefficient sinogram can be obtained as the attenuation coefficient data AD. When the concurrent reconstruction element 24 uses the concurrent reconstruction algorithm for MLAA method, the data of the attenuation coefficient image can be obtained as the attenuation coefficient data AD. Once the concurrent reconstruction processing is completed, the concurrent reconstruction element 24 sends the attenuation coefficient data AD to the attenuation correction element 25.

Step P3 (Attenuation Correction Processing)

Once the attenuation coefficient data AD is sent to the attenuation correction element 25, referring to FIG. 8, the attenuation correction element 25 reads out the concurrent counting data DS relative to the living body S from the memory storage 11. In addition, the attenuation correction element 25 may directly send the concurrent counting data DS from the data collection element 23. And the attenuation correction element 25 executes the attenuation correction processing for the concurrent counting data DS using the estimated attenuation coefficient data AD. The PET image TS of the living body S is reconstructed by the attenuation correction processing that the attenuation correction element 25 executes. The PET image TS is the image that denotes distribution of the radioactive pharmaceuticals of the living body S and on which the attenuation correction is executed relative to the entire living body S.

The attenuation correction processing executed by the attenuation correction element 25 in the second mode, as the same as the attenuation correction processing that the attenuation correction element 22 executes in the first mode, can be the reconstruction operation following the attenuation coefficient correction or the reconstruction operation while executing the attenuation coefficient correction. Specifically, the transmittance of the γ-ray is calculated from the attenuation coefficient data AD estimated by the concurrent reconstruction element 24 and the correction in which the transmittance of the γ-ray is divided from the concurrent counting data DS is executed followed by the corrected concurrent counting data DS may be reconstructed to generate the PET image TS. In addition, the attenuation correction element 25 executes the correction to provide the reconstruction image (PET image TS) from which an effect of γ-ray attenuation is eliminated by incorporating the estimated attenuation coefficient data AD into the calculating express that is used for reconstructing the concurrent counting data DS.

Given the examination subject is the living body S, the attenuation coefficient data AD, which are obtained by using the concurrent reconstruction algorithm, are the data denoting the distribution of the attenuation coefficient relative to the entire living body S. Therefore, when the examination subject is the living body S, the concurrent counting data DS are reconstructed using the attenuation coefficient data AD, so that the PET image TS, on which the adequate attenuation correction relative to the entire living body S is executed, can be generated.

Step P4 (Display of the PET Image)

The data of the PET image TS are sent to the display element 9 and the memory storage 11. The display element 9 displays the PET image and the memory storage 11 stores the data of the PET image TS. The operator conducts an diagnosis as to the distribution of the radioactive pharmaceuticals in the living body S by using the data of the PET image TS. The imaging processing of the PET image is completed by processings of Step P1 or Step P4 in the case of the living body S that is specified as the examination subject.

Effects of the Aspect of Embodiment

(Term 1) A diagnostic apparatus 1 for nuclear medicine, according to the present embodiment, is a diagnostic apparatus for nuclear medicine that generates a radiation image of a phantom using a radioactive pharmaceutical that emits a pair of annihilation radiations comprises: a memory element 11 that stores respectively a template radiation image RA denoting distribution of the radioactive pharmaceuticals in the phantom F, which is obtained in advance, and an entire attenuation coefficient image FP denoting distribution of attenuation coefficients of the entire phantom F; a detection ring that detects a pair of annihilation radiations; a collection element 18 that collects concurrent counting data DK based on the information of the pair of annihilation radiations detected by the detector ring 2; a reconstruction element 19 that generates an actual measurement radiation image GR by reconstructing the concurrent counting data DK; a position deviation calculation element 20 that calculates a difference between a position of the phantom when generating the actual measurement radiation image GR and a position of the phantom when obtaining the template radiation image as a position deviation level RS; a position correction element 21 that generates a corrected attenuation coefficient image HF by correcting the position of the phantom F incorporated in the entire attenuation coefficient image FP based on the position deviation level RS; and a first attenuation correction element (22) that reconstructs the corrected radiation image (TF), which is subjected to the attenuation correction and denoting the distribution of the radioactive pharmaceuticals in the phantom F, by executing an attenuation correction processing using the corrected attenuation coefficient image HF and the concurrent counting data DK.

According to the diagnostic apparatus for nuclear medicine of the Term 1, the template radiation image RA and the entire attenuation coefficient image FP are obtained in advance, and such two images are stored in the memory storage in advance. And the estimation of the attenuation coefficient image is executed using the preliminarily stored template radiation image RA and the entire attenuation coefficient image FP every time when the radiation image as to the phantom F is generated, Specifically, the concurrent counting data DK relative to the phantom F subjected to the examination are obtained, and the actual measurement radiation image GR is generated by reconstructing the concurrent counting data DK. And the position deviation level RS between the actual measurement radiation image GR and the template radiation image RA is calculated, and then the preliminarily obtained entire attenuation coefficient image FP is corrected based on such a position deviation level RS. The corrected attenuation coefficient image HF that is used in the attenuation correction processing relative to the obtained concurrent counting data DK relative to the phantom F is generated by correcting the entire attenuation coefficient image FP.

Here the inventors set forth the effect of the diagnostic apparatus for nuclear medicine according to the aspect of the present Embodiment while comparing with conventional configurations. According to the conventional general PET apparatus, when the attenuation correction processing is executed for the concurrent counting data obtained relative to the examination subject, transmission data or an X-ray CT image is needed. However, according to the conventional configuration, an external radiation source that irradiates the radiation from which is the same kind of nuclide as the positron emission nuclide or X-rays therefrom to the examination subject must be equipped with the PET apparatus. Therefore, it is problematic that the apparatus per se must be made bigger and the cost thereof must increase.

The method that estimate the attenuation coefficient data using the concurrent reconstruction algorithm is proposed, wherein the attenuation correction processing for the concurrent counting data is executable structurally without using the external radiation source. Whereas, according to the keen examination of the inventors, it has been found that the configuration in which the attenuation coefficient is estimated using the concurrent reconstruction algorithm in the case of that the examination subject is the phantom impairs the degree of the precision of the attenuation correction processing for the concurrent counting data. Specifically, referring to FIG. 9B, the concurrent reconstruction algorithm is incapable of estimating the attenuation coefficient data (μ-map) relative to the entire phantom F in principle, so that the attenuation correction processing cannot be executed on at least a part of the phantom F. Therefore, according to the configuration in which the concurrent reconstruction algorithm is applied, it is newly problematic that the degree of precision of the attenuation correction processing relative to the non-clinical evaluation using the phantom impairs.

Further, the inventors examined and considered the results in which the method of executing the attenuation correction processing applying such a μ-map to the PET image of the phantom generated by using the PET apparatus has come up as the method of estimation of the attenuation coefficient relative to the entire phantom without using the external radiation source following obtaining the μ-map of the entire phantom using the X-ray CT apparatus in advance. Regardless of such a method, it is too hard to execute the attenuation correction processing with the high degree of precision. Specifically, the position of the phantom when generating the μ-map and the position of the phantom when generating the PET image are different each other because the X-ray CT apparatus that is used to obtain the μ-map of the entire phantom is a different apparatus from the PET apparatus that is used to generate the PET image of the phantom. Accordingly, the degree of precision of the attenuation correction processing impairs due to the fact that the respective position of the phantoms are not coincident. Specifically, the case in which the attenuation correction processing is executed outside the image of the phantom incorporated in the PET image or the case in which the attenuation correction processing is not executed on at least a part of the phantom image takes place, so that the degree of precision of the attenuation correction processing can be least likely improved.

The diagnostic apparatus for nuclear medicine according to the aspect of the present Embodiment stores the template radiation image RA and the entire attenuation coefficient image FP that are preliminarily obtained. Next, the actual measurement radiation image GR is generated by reconstructing the concurrent counting data DK that are collected relative to the phantom F at present time (Step S1, S2). And the corrected attenuation coefficient image HF is estimated by correcting the position of the phantom incorporated in the entire attenuation coefficient image FP based on the position deviation level RS between the actual measurement radiation image GR and the template radiation image RA (Step S3, S4). Further the PET image TF on which the attenuation correction is executed is reconstructed by executing the attenuation correction processing for the concurrent counting data DK using the estimated and corrected attenuation coefficient image HF.

The template radiation image RA is the image denoting the radiation distribution in the phantom and the entire attenuation coefficient image FP is the image wherein the attenuation coefficient distribution relative to the entire phantom is estimated. Therefore, the corrected attenuation coefficient image HF, which is obtained by correcting the position of the phantom F, which is incorporated in the entire attenuation coefficient image FP, is also the image wherein the distribution of the attenuation coefficient is estimated in the entire phantom. Therefore, the attenuation correction processing is executed relative to the entire area of the phantom F including the housing 41 by using the estimated and corrected attenuation coefficient image HF without limiting the area of the living body dummy 43 in which the radioactive pharmaceuticals are diffusible.

And the position correction element 21 estimates the corrected attenuation coefficient image HF by correcting the position of the phantom incorporated in the entire attenuation coefficient image FP according to the position deviation level between the template radiation image RA and the actual measurement radiation image GR. Specifically, the position of the attenuation coefficient distribution relative to the corrected attenuation coefficient image HF is corrected so as to correspond to the position of the phantom image incorporated in the actual measurement radiation image GR by the position correction element 21. Therefore, the attenuation correction element 22 becomes capable of executing the attenuation correction processing by the corrected attenuation coefficient image HF as for the concurrent counting data DK collected when generating the actual measurement radiation image GR.

And the corrected attenuation coefficient image HF is the image in which the attenuation correction distribution of the entire phantom is estimated, so that the PET image TF generated by the attenuation correction element 22 is the radiation image on which the attenuation correction is executed relative to the entire phantom F. Accordingly, the PET image TF of the phantom F, for which the attenuation correction processing is executed with a high degree of precision, is obtained without equipping the external radiation source with the PET apparatus 1. Specifically, an accurate attenuation correction processing relative to the concurrent counting data can be improved in the non-clinical evaluation using the phantom can be executed while avoiding the PET apparatus 1 to be bigger.

(Term 2) The nuclear diagnostic apparatus according to Term 1, comprises the mode switching element 13 that switches to select the first mode when the examination subject is the phantom F and the second mode when the examination subject is the living body S; the concurrent reconstruction element 24 that generates the radiation distribution image ER that denotes the distribution of the radioactive pharmaceuticals in the examination subject and the attenuation coefficient data AD that denotes the distribution of the attenuation coefficient in the area at which the radioactive pharmaceuticals of the examination subject distribute by executing the concurrent reconstruction processing relative to the concurrent counting data DS using the concurrent reconstruction algorithm; and the second attenuation correction element that reconstructs the corrected image TS that is subjected to the attenuation correction and denotes the distribution of the radioactive pharmaceuticals relative to the examination subject by executing using the attenuation coefficient data AD and the concurrent counting data DS. Wherein when the mode switching element is switched to the first mode, it is controlled so that the reconstruction element generates the actual measurement radiation image, the deviation calculation element calculates the position deviation level, the position correction element generates the corrected attenuation coefficient image, and the first attenuation correction element reconstructs the corrected radiation image, and when the mode switching element is switched to the second mode, it is controlled so that the concurrent reconstruction element generates the radiation distribution image and the attenuation coefficient data.

The diagnostic apparatus for nuclear medicine according to Term 2 comprises the mode switching element 13 corresponding to the examination subject. When the examination subject is the phantom, the mode switching element 13 switches the operation of the diagnostic apparatus for nuclear medicine to the first mode, and when the examination subject is the living body, the mode switching element 13 switches the operation of the diagnostic apparatus for nuclear medicine to the second mode.

When switched to the first mode, as well as the diagnostic apparatus for nuclear medicine according to Term 1, the corrected radiation image of the phantom is generated by the reconstruction element, the position deviation calculation element, the position correction element and the first attenuation correction element. Specifically, the template radiation image RA and the entire attenuation coefficient image FP are obtained in advance, and such two images are stored in the memory storage 11 in advance. And the estimation of the attenuation coefficient image is executed using the preliminarily stored template radiation image RA and the entire attenuation coefficient image FP every time when the radiation image as to the phantom F is generated. Therefore, even when the examination subject is the phantom, the radiation image on which the accurate attenuation correction processing relative to the entire phantom is executed can be obtained without using the external radiation source.

On the other hand, when switched to the second mode, the concurrent reconstruction element and the second attenuation correction element are used to generate the corrected image of the living body S. The reconstruction element can estimate the attenuation coefficient data AD from the concurrent counting data DS obtained relative to the living body S by using the concurrent reconstruction algorithm.

The second attenuation correction element executes the attenuation correction processing relative to the concurrent counting data DS using the attenuation coefficient data AD and generates the corrected image that denotes the distribution of the radioactive pharmaceuticals in the living body S. The position of the living body S incorporated in the attenuation coefficient data AD corresponds to the position of the living body when obtaining the concurrent counting data DS, so that the attenuation correction processing relative to the concurrent counting data DS can be executed using the attenuation coefficient data AD. When the examination subject is the living body S, the radioactive pharmaceuticals are diffused in the entire living body. Therefore, the attenuation coefficient data AD that denotes the distribution of the attenuation coefficients in the entire living body S can be generated by the operation processing using the concurrent reconstruction algorithm.

Accordingly, the corrected image (PET image TS) can be obtained as the radiation image on which the accurate attenuation correction is executed relative to the living body S by using the attenuation coefficient data AD. Specifically, the diagnostic apparatus for nuclear medicine that executes the accurate attenuation correction relative to the entire examination subject can be brought in reality without equipping the external radiation source as to respective both the case in which the examination subject is the living body and the case in which the examination subject is the phantom.

(Term 3) According to the diagnostic apparatus for nuclear medicine described in Term 2, the entire attenuation coefficient image FP can be obtained by estimating the distribution of the attenuation coefficients in the entire phantom F using the geometric information of the phantom F.

According to the diagnostic apparatus for nuclear medicine described in Term 3, the entire attenuation coefficient image FP can be obtained by estimating the distribution of the attenuation coefficients in the entire phantom F using the geometric information of the phantom F. The respective members of the phantom F can preliminarily get the information such as the position that is in place in the phantom and the construction materials therefor. The attenuation of the γ-rays can be preliminarily calculated provided the construction material is already known, so that the distribution of the attenuation coefficient in the entire phantom F can be preliminarily calculated by the geometric operation method without actually irradiating radiations to the phantom F by knowing preliminarily the type of construction material and the position thereof. Accordingly, the entire attenuation coefficient image FP can be preliminarily obtained without using another e.g., an X-ray CT apparatus or a PET/CT apparatus other than the PET apparatus 1.

(Term 4) The estimation method of the attenuation coefficient image according to the aspect of the present Embodiment is the estimation method of the attenuation coefficient image that estimates an attenuation coefficient image of the phantom in the diagnostic apparatus for nuclear medicine comprises; a memory storing step of storing a template radiation image RA, which is obtained in advance, denoting distribution in the phantom of the radioactive pharmaceutical that emits a pair of an annihilation radiations and an entire attenuation coefficient image FP denoting distribution of the attenuation coefficient in the entire phantom in the memory storage; a detection step of detecting the pair of annihilation radiations emitted from the inside of the phantom using detection rings 2 which are in place in the positions surrounding the phantom; a data collection step of collecting concurrent counting data DK based on the information of the pair of annihilation radiations detected in the detection step; a reconstruction step of generating an actual measurement radiation image by reconstructing the concurrent counting data DK; a position deviation calculation step of calculating a difference between a position of the phantom F when obtaining an actual measurement radiation image GR and a position of the phantom F when obtaining the template radiation image as a position deviation level RS; and a position correction step of estimating a corrected attenuation coefficient image HF by correcting the position of the phantom F incorporated in the entire attenuation coefficient image FP based on the position deviation level RS that is calculated in the position deviation calculation step.

According to the estimation method of the attenuation coefficient described in Term 4, the template radiation image and the entire attenuation coefficient image are stored in the template data storing step. And the concurrent counting data based on the pair of annihilation radiations emitted from the inside phantom is collected during the detection step and the data collection step. In the reconstructing step, the actual measurement radiation image is generated by reconstructing the concurrent counting data. In the position deviation calculation step, the difference between the position of the phantom on obtaining the actual measurement radiation image and a position of the phantom on obtaining and position of the phantom on obtaining the template radiation image is calculated as a position deviation level. In the position correction step, the corrected attenuation coefficient image is estimated by correcting the position of the entire attenuation coefficient image based on the position deviation level.

The template radiation image RA is the image denoting the radiation distribution in the phantom and the entire attenuation coefficient image FP is the image wherein the attenuation coefficient distribution relative to the entire phantom is estimated. Therefore, the corrected attenuation coefficient image HF, which is obtained by correcting the position of the phantom F incorporated in the entire attenuation coefficient image FP, is also the image wherein the distribution of the attenuation coefficient is estimated in the entire phantom. Therefore, the attenuation correction processing is executed relative to the entire area of the phantom F by using corrected attenuation coefficient image HF without limiting the area where the radioactive pharmaceuticals are diffusible.

And in the position correction step, the corrected attenuation coefficient image HF is estimated by correcting the position of the phantom incorporated in the entire attenuation coefficient image HF according to the position deviation level between the template radiation image RA and the actual measurement radiation image GR. Specifically, the position of the attenuation coefficient distribution relative to the corrected attenuation coefficient image HF is corrected so as to correspond to the position of the phantom image incorporated in the actual measurement radiation image GR by the position correction element 21. Therefore, the attenuation correction processing can be executed by the corrected attenuation coefficient image HF relative to the concurrent counting data DK collected when generating the actual measurement radiation image GR. Accordingly, the corrected attenuation coefficient image HF capable of executing the attenuation correction processing for the concurrent counting data DK of the phantom F can be estimated without using the external radiation source. Specifically, an accurate attenuation correction processing relative to the concurrent counting data can be executed in the non-clinical evaluation using the phantom while avoiding that the diagnostic apparatus for nuclear medicine must be bigger.

(Term 5) According to the diagnostic apparatus for nuclear medicine described in Term 4, the entire attenuation coefficient image can be obtained by estimating the distribution of the attenuation coefficients in the entire phantom F using the geometric information of the phantom.

According to the diagnostic apparatus for nuclear medicine described in Term 5, the entire attenuation coefficient image FP can be obtained by estimating the distribution of the attenuation coefficients in the entire phantom F using the geometric information of the phantom F. In such a case, the distribution of the attenuation coefficient in the entire phantom F can be preliminarily calculated by the geometric operation method without actually irradiating radiations to the phantom F. Accordingly, the entire attenuation coefficient image FP can be preliminarily obtained without using another e.g., an X-ray CT apparatus or a PET/CT apparatus other than the PET apparatus 1.

### Other Embodiment

Specifically, Embodiments disclosed at this time are examples in all aspects and not limited thereto. The scope of the present invention includes what claims is claimed and all alternatives and equivalents thereof within the scope thereof. For example, the present invention can be implemented in the below alternative Embodiment.

(1) According to the embodiment set forth above, the living body dummy 43 may not be limited to the model of human brain. The other example of the living body dummy 43 includes the model of the entire living human, a model of a breast of the living human and the model of a creation other than a living human.

(2) According to the embodiment described above, the detection rings 2 have the structure in which three scintillator blocks 31 are layered in the p-direction but the number of the scintillator blocks 31 arrayed in the p-direction can be arbitrarily modified. The detection ring 2 may have the structure in which the scintillator block 31 is a single layer or 2 or moth than 4 scintillator blocks 31 can be layered in the p-direction.

(3) According to the Embodiment described above, the inventors set forth the diagnostic apparatus for nuclear medicine as an example, the PET apparatus is not limited thereto as long as the configuration can estimate the attenuation coefficient image. For an example, the invention can be applied to such as SPECT apparatus (Single Photon Emission Computed Tomography) apparatus.

(4) According to the embodiment described above, the γ-ray detector 3 is not limited to the configuration illustrated in FIG. 2 and can be arbitrarily modified. For an example, the γ-ray detector 3 is not limited to the DOI detector and the scintillator element consisting of the scintillator block 31 can be a single layer in the depth direction. And as another example, the γ-ray detector 3 may not have the light guide 32. Further, the γ-ray detector 3 may have the semiconductor element such as SiPM Silicon Photomultiplier) instead of photomultiplier 33.

### REFERENCE LIST

1 PET apparatus
2 Detector ring
3 γ-Ray detector
4 Concurrent counting circuit
5 Main control unit
6 Attenuation coefficient correction estimation program
7 Input element
9 Display element
10 Table
11 Memory storage
13 Mode switching element
15 First image processing unit
17 Second image processing unit
18 Data collection element
19 Reconstruction element
20 Position deviation calculation element
21 Position correction element
22 Attenuation correction element
23 Data collection element
24 Concurrent reconstruction element
25 Attenuation correction element
31 Scintillator block
32 Light guide
33 Photomultiplier
41 Housing
42 Inner side storage element
43 Living body dummy
S Living body
F Phantom
DK Concurrent counting data
DS Concurrent counting data
RA Template radiation image
FP Entire attenuation coefficient image
GR Actual measurement radiation image
HF Corrected attenuation coefficient image
RS Position deviation level
AD Attenuation coefficient data
ER Radiation distribution image
TF PET image
TS PET image

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure covers modifications and variations of this disclosure provided they come within the scope of the appended claims and their equivalents.

What is claimed is:

1. A diagnostic apparatus, for nuclear medicine, that generates a radiation image of a phantom using a radioactive pharmaceutical that emits a pair of annihilation radiations, comprising:
a memory element that stores a template radiation image, which is obtained in advance, denoting distribution of the radioactive pharmaceutical in the phantom and an entire attenuation coefficient image denoting distribution of the attenuation coefficient in the entire phantom;
a detection ring that detects said pair of annihilation radiations;
a data collection element that collects concurrent counting data based on information of said pair of annihilation radiations detected by said detector ring;
a reconstruction element that generates an actual measurement radiation image by reconstructing said concurrent counting data;
a position deviation calculation element that calculates difference between a position of the phantom when generating the actual measurement radiation image and a position of the phantom when obtaining the template radiation image as a position deviation level;
a position correction element that generates a corrected attenuation coefficient image by correcting said position of said phantom incorporated in said entire attenuation coefficient image based on said position deviation level; and
a first attenuation correction element that reconstructs the corrected radiation image, on which the attenuation correction is executed and denoting the distribution of the radioactive pharmaceutical in the phantom, by an attenuation correction processing using the correct attenuation coefficient image and the concurrent counting data.

2. The diagnostic apparatus, for nuclear medicine, according to claim 1, further comprising:
a mode switching element that switches to select a first mode when an examination subject is a phantom F and to a second mode when said examination subject is a living body;
a concurrent reconstruction element that generates a radiation distribution image that denotes distribution radioactive pharmaceuticals in said examination subject and the attenuation coefficient data that denotes distribution of attenuation coefficients in an area at which said radioactive pharmaceuticals of said examination subject distribute by executing a concurrent reconstruction processing relative to said concurrent counting data using a concurrent reconstruction algorithm; and
a second attenuation correction element that reconstructs said corrected image on which an attenuation correction is executed and denotes said distribution of said radioactive pharmaceuticals relative to said examination subject by executing an attenuation correction processing using said attenuation coefficient data of said the concurrent counting data;
wherein when said mode switching element is switched to said first mode, said reconstruct element is controlled so that said reconstruction element generates said actual measurement radiation image, said deviation calculation element calculates said position deviation level, said position correction element generates said corrected attenuation coefficient image, and said first attenuation correction element reconstructs said corrected radiation image, and
wherein when said mode switching element is switched to said second mode, said concurrent reconstruction element is controlled to generate said radiation distribution image and said attenuation coefficient data, and said second attenuation correction element is controlled to generates said corrected image relative to said living body.

3. The diagnostic apparatus, for nuclear medicine, according to claim 1, wherein:
said entire attenuation coefficient image can be obtained by estimating distribution of said attenuation coefficients in said entire phantom using geometric information of said phantom.

4. An estimation method, of an attenuation coefficient image, that estimates an attenuation coefficient image of a phantom in said diagnostic apparatus for nuclear medicine comprising:
a template data memory storing step of storing a template radiation image, which is obtained in advance, denoting distribution in the phantom of the radioactive pharmaceutical that emits a pair of an annihilation radiations and an entire attenuation coefficient image denoting distribution of the attenuation coefficients in the entire phantom in a memory element;
a detection step of detecting said pair of annihilation radiations emitted from inside of said phantom using detection rings which are in place in said positions surrounding said phantom;
a data collection step of collecting concurrent counting data based on information of said pair of annihilation radiations detected in said detection step;
a reconstruction step of generating an actual measurement radiation image by reconstructing said concurrent counting data;
a position deviation calculation step of calculating difference between said position of said phantom when generating said actually measured radiation image and said position of said phantom when obtaining said template radiation image as a position deviation level; and
a position correction step of estimating said corrected attenuation coefficient image by correcting said position of said phantom incorporated in the entire attenuation coefficient image based on the position deviation level that is calculated in the position deviation calculation step.

5. The estimation method, of said attenuation coefficient image, according to claim 4, wherein:
said entire attenuation coefficient image is obtained by estimating distribution of said attenuation coefficients in said entire phantom using said geometric information of said phantom.
